(19) United States
(12) Patent Application Publication
(10) Pub. No.: US 2022/0331289 A1
(43) Pub. Date: Oct. 20, 2022

(54) Pharmaceutical Composition Comprising an Artemisinin Derivative for Nasal or Pulmonary Delivery

(71) Applicant: Cipla Limited, Mumbai (IN)

(72) Inventors: Preeti RAUT, Mumbai (IN); Geena MALHOTRA, Mumbai (IN)

(21) Appl. No.: 17/724,554

(22) Filed: Apr. 20, 2022

Related U.S. Application Data
(63) Continuation of application No. 15/531,241, filed on May 26, 2017, now abandoned.

(30) Foreign Application Priority Data
Nov. 27, 2014 (IN) 3765/MUM/2014

(51) Int. Cl. A61K 31/357; A61K 9/00; A61K 47/26; A61K 9/19; A61K 45/06; A61K 31/4709; A61K 31/505; A61K 31/635; A61K 31/66; A61K 31/47

(52) U.S. Cl. CPC A61K 31/357; A61K 9/0043; A61K 47/26; A61K 9/19; A61K 45/06; A61K 31/4709; A61K 31/505; A61K 31/635; A61K 31/66; A61K 31/47; A61K 9/007; Y02A 50/30

(57) ABSTRACT

The present invention relates to pharmaceutical composition for intranasal or pulmonary delivery, wherein the composition comprises an artemisinin derivative and optionally one or more pharmaceutically acceptable excipients. The pharmaceutical composition may be for intranasal delivery, may be in the form of a nasal spray, a solution, a suspension, nasal drops, an insufflation powder or a nasal powder, and may be suitable for delivery using a nebulizer, insufflator, powder sprayer or powder inhaler. Alternatively, the pharmaceutical composition may be for pulmonary delivery, may be in the form of an aerosol composition or a powder, and may be suitable for delivery using a metered dose inhaler (MDI) or a dry powder inhaler (DPI). The present invention also relates to processes for preparing such compositions and to the use of such compositions for the treatment of malaria.

### CROSS REFERENCE TO RELATED APPLICATIONS

This application is a continuation application and claims priority to U.S. patent application Ser. No. 15/531,241 filed May 26, 2017, entitled “Pharmaceutical Composition Comprising an Artemisinin Derivative for Nasal or Pulmonary Delivery” which claims priority to International Application No. PCT/GB2015/053624 filed Nov. 27, 2015, entitled “Pharmaceutical Composition Comprising an Artemisinin Derivative for Nasal or Pulmonary Delivery” which claims priority to Indian Patent Application No. 3765/MUM/2014 filed Nov. 27, 2014, which applications are incorporated by reference herein in their entirety.

### FIELD OF INVENTION

The present invention relates to a pharmaceutical composition comprising an artemisinin derivative, a process for preparing such a pharmaceutical composition and its use in the treatment of malaria.

### BACKGROUND AND PRIOR ART

Malaria remains a tremendous health burden in tropical areas causing up to 24.3 billion episodes of clinical illness and 0.86 million deaths, with annual death rates of up to 93% affected by severe malaria. A small proportion of children may also suffer from long-term neurological disability as a consequence of repeated bouts of severe malaria. Severe malaria occurs when infection with the P. falciparum parasite is complicated by serious organ failure or metabolic abnormalities; cerebral malaria, an unarousable coma not attributable to any other cause, is a specific type of severe malaria that even with appropriate treatment can have a mortality rate approaching 20%. A small proportion of cerebral malaria survivors are left with persistent neurological sequelae. Severe malaria occurs most commonly in those with limited immunity to malaria. In highly endemic areas, young children are therefore at most risk of severe disease and death, whereas in areas of lower endemicity, travelers, both adults and children, get severe disease.

Chloroquine (CQ) or quinine has been both an affordable and well-tolerated drug for nearly 400 years. Despite its long history of efficacy, quinine has significant limitations. Even with prompt administration, case fatality rates in severe malaria often exceed 20%. Adverse effects resulting from quinine therapy are unfortunately common. Cinchonism often occurs at conventional dose regimens. This usually mild and reversible symptom complex consists of tinnitus, deafness, dizziness, and vomiting, and may affect patient adherence. Hypoglycaemia is a less common, but more serious, adverse effect. Some people are allergic to quinine and develop skin rashes and edema, even with small doses. Toxic levels of quinine can occur following rapid intravenous administration and can result in heart rhythm disturbances, blindness, coma, and even death; hence routine cardiac monitoring during parenteral treatment is always recommended. But chloroquine now faces severe limitations due to widespread CQ resistant P. falciparum strains and, a few reports of P. vivax strains. To overcome this problem, different combinations of antimalarial drugs have been used, but in most instances, multidrug-resistant P. falciparum strains have emerged.

Quinidine is the only U.S. FDA approved drug for treatment of severe malaria. Though it is commercially available and effective against malaria, it is not an ideal drug. Like quinine, quinidine is also associated with sudden cardiac death, principally via cardiac arrhythmias, and, because of its short half-life, must be administered 2-3 times a day. Quinidine is more cardiotoxic than quinine and is always administered in an intensive care unit with continuous electrocardiographic and frequent blood pressure monitoring. Quinidine-related cardiovascular adverse effects are potentially serious and may be more frequent if the drug is administered rapidly. The risk of cardiotoxicity is increased with bradycardia, hypokalemia, and hypomagnesemia and if the patient has received other drugs that may prolong the QTc interval (e.g., quinine, mefloquine, or macrolide antibiotics).

Artemisinin derivatives on the other hand, for the last two decades, have been quite effective in clearing the parasitaemia, within 48 to 72 hours. Artemisinin is a natural component of the plant Artemisia annua, concoctions of which have been used for a very long time in traditional Chinese medicine for the treatment of fever. In 1972, the component responsible for the pharmacodynamics action, qinghaosu or artemisinin, was isolated from the leaves of this plant, and its activity against the malaria parasite Plasmodium falciparum was subsequently demonstrated. A number of semi-synthetic derivatives were also prepared for use in malaria combat programmes. Best known among the different derivatives are artemether, arteether (artemotil), artesunate and artenimol (β-dihydroartemisinin, DHA). The biological activity of artemisinin and its derivatives is based on the reactivity of the endoperoxide bridge, the common structural feature of artemisinin and all of its derivatives.

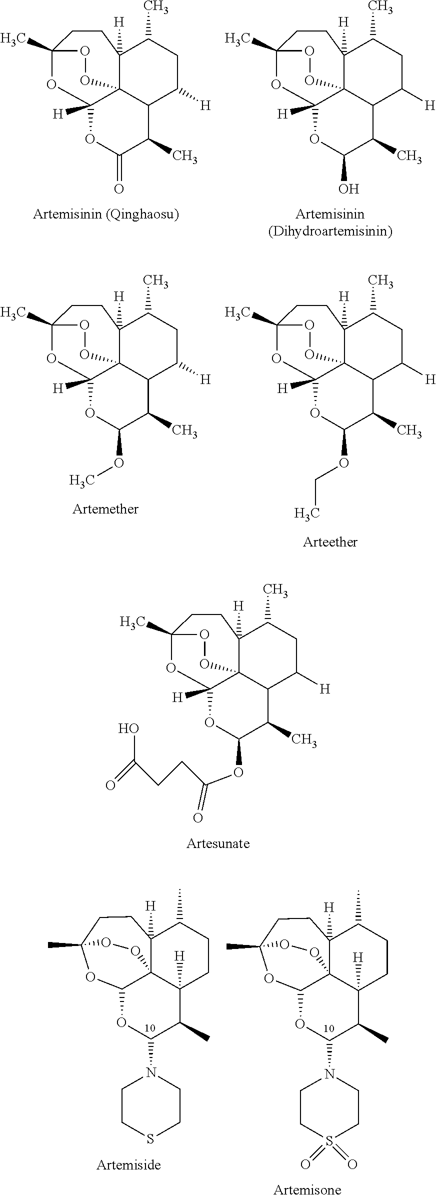

Artemisinin derivatives are fast acting substances, leading to a rapid clearance of the malarial parasites from the blood, while the short biological half-life precludes a long-lasting activity. Artemisinin and its derivatives have been shown to act very efficiently against the asexual, erythrocytic forms of Plasmodium falciparum (from the early ring stages to the schizontes); as well as its activity has also been demonstrated against P. vivax.

The various derivatives do have antimalarial activity of their own, but the main therapeutic efficacy is due to the (rapidly occurring) biotransformation into the primary metabolite, artenimol (β-dihydroartemisinin, DHA), which is considered to be the ultimate active agent.

The generally recommended effective oral dose in humans with uncomplicated falciparum malaria ranges between 10 mg/kg/day for artemisinin and 2-5 mg/kg/day for artemisinin derivatives.

When artemisinins are used as monotherapy, a minimum of a seven day course of therapy is required to prevent recrudescence. However, for regimens of less than seven days a combination with another effective blood schizonticide is necessary.

Artesunate is commercially available as an oral tablet formulation, rectal capsules and as an injectable preparation, which can be administered either intravenously or intramuscularly. The injectable preparation is mostly preferred over the oral dosage forms as it achieves therapeutic plasma concentrations rapidly when administered by either an intravenous or intramuscular route.

WO 2010/110747 discloses artemisinin derivatives in the treatment of an airway disorder such as asthma and chronic obstructive pulmonary disease.

EP 2424523 B discloses sublingual spray formulation comprising dihydroartemesinin for the treatment of neoplasms.

Treatment of malaria in a mouse model by intranasal drug administration, Elka Touitou et al, International Journal for Parasitology 36 (2006) 1493-1498. This article concludes that the treatment and prophylaxis with intranasal dihydroartemisinin DHA was effective in ameliorating Plasmodium infection in a rodent model of severe malaria. The intranasal delivery system contains DHA in a lipid carrier composed of phospholipid fluid vesicles.

Design and in vitro evaluation of nanoemulsion for nasal delivery of artemether, Hitendra S. Mahajan et al, Indian Journal of Novel Drug delivery 3(4), Oct-Dec, 2011, 272-277. This article concludes that artemether containing nanoemulsion formulation was successfully prepared by spontaneous emulsification method (titration method) and was feasible for nasal administration, and was expected to rapidly exert its antimalarial effect.

Although all the above prior art documents disclose that artemisinin derivatives can be administered for the treatment of malaria, none of the above mention them as being administered in the form of compositions which are easy to manufacture, cost effective and which may be useful in rapidly reducing the parasite bio-mass of an infected patient, thereby increasing the patient's chance of recovery and patient compliance while decreasing treatment costs.

Hence, there is a need to develop a pharmaceutical composition comprising an artemisinin derivative which would be economical, easy to manufacture as well as ensure fast recovery and patient compliance.

### OBJECT OF THE INVENTION

An object of the present invention is to provide a pharmaceutical composition comprising an artemisinin derivative optionally with one or more pharmaceutically acceptable excipients.

Another object of the present invention is to provide a pharmaceutical composition comprising an artemisinin derivative to ensure patient compliance.

Yet another object of the present invention is to provide a pharmaceutical composition comprising an artemisinin derivative with reduced dose.

Another object of the present invention is to provide a pharmaceutical composition comprising an artemisinin derivative with increased bioavailability.

Yet another object of the present invention is to provide a process of preparing a pharmaceutical composition comprising an artemisinin derivative and optionally with one or more pharmaceutically acceptable excipients.

A further object of the present invention is to provide a method of treating malaria which method comprises administering a pharmaceutical composition comprising an artemisinin derivative and optionally with one or more pharmaceutically acceptable excipients.

Another object of the present invention is to provide a pharmaceutical composition comprising an artemisinin derivative and optionally with one or more pharmaceutically acceptable excipients for use in the treatment of malaria.

The invention relates to new formulations providing an artemisinin derivative via the intranasal or pulmonary route. These formulations demonstrate good bioavailability (rate and extent of absorption) and do not show any toxicity/irritation towards the nasal epithelial membranes and ciliary movement.

### SUMMARY OF THE INVENTION

According to one aspect of the present invention, there is provided a pharmaceutical composition comprising an artemisinin derivative and optionally one or more pharmaceutically acceptable excipients.

According to another aspect of the present invention, there is provided a process of preparing a pharmaceutical composition comprising an artemisinin derivative and optionally one or more pharmaceutically acceptable excipients.

According to a further aspect of the present invention there is provided a method of treating malaria by administering a pharmaceutical composition comprising an artemisinin derivative.

According to yet another aspect of the present invention, there is provided the use of a pharmaceutical composition comprising an artemisinin derivative in the manufacture of a medicament for treating malaria.

### BRIEF DESCRIPTION OF DRAWINGS

FIG. 1 depicts concentration v/s time curve for DHA after intravenous and intranasal administration of artesunate formulations (6.25 mg/kg) in rabbits.

### DETAILED DESCRIPTION OF THE INVENTION

Some of the artemisinin derivatives, especially artesunate, are poorly soluble in water as well as exhibiting poor stability in aqueous solutions at neutral or acidic pH. Hence artesunate is commercially available as an injectable powder formulation which needs to be reconstituted with two different solvents such as sodium bicarbonate and glucose solution or sodium chloride prior to administration either by an intravenous or intramuscular route. Further, for each and every such administration, artesunate solution needs to be freshly prepared and the remaining unused solution has to be discarded as it cannot be stored.

Further, rectal suppositories of artesunate are being looked at as an alternative route of administration to the existing intravenous and intramuscular routes, with the aim of providing safe and effective treatment of severe malaria in rural areas of tropical regions. However, such rectal suppositories may not be able to cure the disease, i.e. they may not be able to completely eliminate the malarial parasites from the blood.

Another artemisinin derivative, artemether, is commercially available as an oil-based intramuscular injection and as oral tablets. But, the injectable preparation of artemether is mostly preferred over the oral dosage forms as it achieves therapeutic plasma concentrations rapidly. However, the oil-based injection formulation is slowly and erratically absorbed, with relatively little conversion to the active metabolite dihydroartemisinin (DHA) Further, such oil-based injections can be uncomfortable for the patient as they cause severe pain during administration.

Generally, the tablet dosage form is the most preferable dosage form, but for certain therapies such as malaria, parenteral dosage forms are recommended as they achieve rapid therapeutic plasma concentrations. However, the commercially available parenteral formulations of artemisinin derivatives have not been able to completely address the drawbacks that are associated with the parenteral dosage form of existing artemisinin derivatives such as slow and erratic absorption, and poor stability, as well as the drawbacks that are associated with patient compliance.

The present invention thus provides a pharmaceutical composition comprising an artemisinin derivative which rapidly reduces the parasite bio-mass of an infected patient and that will ensure patient compliance.

The nasal mucosa is a potential route of drug administration for achieving faster and higher levels of absorption. This is mainly due to the large surface area of the nasal mucosa, its porous endothelial membrane, the high amount of blood flow in the mucosa and its ability to bypass the first-pass hepatic metabolism. The nasal cavity is covered by a thin mucosa which is well vascularized, therefore, drugs can be transferred quickly across the single epithelial cell layer directly to the systemic blood circulation without undergoing the first-pass hepatic metabolism or the intestinal metabolism. Further, the therapeutic effect after nasal administration is achieved very quickly for smaller drug molecules as compared to the oral route of administration.

The term “artemisinin derivative” is used in a broad sense to include not only “artesunate”, “artemether”, “dihydroartemisinin”, “artemisone”, “arteether”, “artenimol”, “artesunic acid”, “artelinic acid”, “deoxoaretmisinin”, “artemotil” and “artemiside” per se but also their pharmaceutically acceptable salts, pharmaceutically acceptable solvates, pharmaceutically acceptable hydrates, pharmaceutically acceptable enantiomers, pharmaceutically acceptable esters, pharmaceutically acceptable polymorphs, pharmaceutically acceptable prodrugs, pharmaceutically acceptable complexes etc.

It will be understood, that the specific dose level and frequency of dosage according to the invention for any particular patient may be varied and will depend upon a variety of factors including the activity of the specific compound employed, the metabolic stability and length of action of that compound, the patient's age, body weight, general health, sex and diet, the mode and time of administration, the rate of excretion, the drug combination, the severity of the particular condition, and the host undergoing therapy.

In an embodiment, the artemisinin derivative may be administered in a daily dose of from about 2 mg/kg of body weight to about 9 mg/kg of body weight to a patient.

The term “pharmaceutical composition” includes metered dose inhalers (MDI), dry powder inhalers (DPI), nebulizers, nasal sprays, nasal drops, insufflation powders or nasal powders and the like. The pharmaceutical compositions of the present invention may be administered by any suitable methods used for delivery of the drugs to the systemic circulation.

The pharmaceutical compositions of the present invention are formulated for intranasal or pulmonary delivery and may therefore be administered by any suitable methods used for delivery of the drugs to the nasal mucosa or lungs. For example, the composition of the present invention may be in the form of aerosol compositions, powders, sprays, solutions, suspensions, drops, an insufflation powder or nasal powder. Such compositions may be administered by any conventional means, for example using a metered dose inhaler (MDI), a dry powder inhaler (DPI), a nebulizer, an insufflator, a powder sprayer or a powder inhaler.

Preferably, the pharmaceutical composition is presented in the form of a powder dosage form for inhalation and may be administered using a dry powder inhaler, a nasal insufflator, a nasal powder sprayer or a powder inhaler. Most preferably, the pharmaceutical composition is administered using a nasal insufflator, a nasal powder sprayer or a powder inhaler.

The various dosage forms according to the present invention may comprise pharmaceutically acceptable excipients suitable for formulating the same.

Pharmaceutically acceptable excipients suitable for use with pharmaceutical compositions for intranasal delivery include a carrier, a solvent, a vehicle, a thickening agent, a tonicity agent, a pH regulator, a chelating agent, or combinations thereof.

Suitable carriers that may be incorporated in the pharmaceutical composition include, but are not limited to, sugars such as glucose, saccharose, lactose and fructose, saccharides, disaccharides, amino acids such as but not limited to, glycine, leucine, isoleucine, arginine, starches or starch derivatives, oligosaccharides such as dextrins, cyclodextrins and their derivatives, polyvinylpyrrolidone, alginic acid, tylose, silicic acid, organic salts such as but not limited to, sodium citrate, ammonium acetate, cellulose, cellulose derivatives (for example cellulose ether), sugar alcohols such as mannitol or sorbitol, calcium carbonate, calcium phosphate, etc. lactose, lactitol, dextrates, calcium stearate, dextrose, maltodextrin, saccharides including monosaccharides, disaccharides, polysaccharides; sugar alcohols such as arabinose, ribose, mannose, sucrose, trehalose, maltose, dextran, magnesium stearate, cellobiose octaacetate and the like or combinations thereof.

In the context of the present invention, the term solvent means that which when added provides a formulation in which the medicament can be dissolved or dispersed.

According to the present invention, the solvent may comprise one or more of, C2-C6 aliphatic alcohols, such as, but not limited to, ethanol, methanol and isopropyl alcohol; water, acetone, glycols such as but not limited to propylene glycol, polyethylene glycols, polypropylene glycols, glycol ethers, and block copolymers of oxyethylene and oxypropylene; and other substances, such as, but not limited to, glycerol, polyoxyethylene alcohols, and polyoxyethylene fatty acid esters; hydrocarbons such as, but not limited, to n-propane, n-butane, isobutane, n-pentane, iso-pentane, neo-pentane, and n-hexane; and ethers such as but not limited to diethyl ether and the like or combinations thereof.

In an alternate embodiment, the pharmaceutical compositions of the present invention are in a form suitable for pulmonary delivery using a metered dose inhaler (MDI), for example, in the form of an aerosol composition. Such compositions may comprise one or more pharmaceutically acceptable excipients, in particular selected from the group of an HFC/HFA propellant, a co-solvent, a bulking agent, a non-volatile component, a buffer/pH adjusting agent, a surfactant, a preservative, a complexing agent, or combinations thereof.

Suitable propellants are those which, when mixed with the solvent(s), form a homogeneous propellant system in which a therapeutically effective amount of the medicament can be dissolved. The HFC/HFA propellant must be toxicologically safe and must have a vapor pressure which is suitable to enable the medicament to be administered via a pressurized MDI.

According to the present invention, the HFC/HFA propellants may comprise, one or more of 1,1,1,2-tetrafluoroethane (HFA-134(a)) and 1,1,1,2,3,3,3,-heptafluoropropane (HFA-227), HFC-32 (difluoromethane), HFC-143(a) (1,1,1-trifluoroethane), HFC-134 (1,1,2,2-tetrafluoroethane), and HFC-152a (1,1-difluoroethane) or combinations thereof and such other propellants which may be known to the person having a skill in the art.

Suitable surfactants which may be employed in an aerosol composition of the present invention include those which may serve to stabilize the solution formulation and improve the performance of valve systems of the metered dose inhaler. Preferred surfactants include one or more ionic and/or non-ionic surfactants. Examples of suitable surfactants include, but are not limited to, oleic acid, sorbitan trioleate, lecithin, isopropylmyristate, tyloxapol, polyvinylpyrrolidone, polysorbates such as polysorbate 80, vitamin E-TPGS, and macrogol hydroxystearates such as macrogol-15-hydroxystearate and the like or combinations thereof.

In the context of the present invention, the term “non-volatile component” refers to the suspended or dissolved constituents of the pharmaceutical composition that would remain after evaporation of the solvent(s) present.

The non-volatile component may comprise one or more of monosaccharides such as, but not limited to, glucose, arabinose; disaccharides such as lactose, maltose; oligosaccharides and polysaccharides such as, but not limited to, dextrans; polyalcohol such as, but not limited to, glycerol, sorbitol, mannitol, xylitol; salts such as, but not limited to, potassium chloride, magnesium chloride, magnesium sulphate, sodium chloride, sodium citrate, sodium phosphate, sodium hydrogen phosphate, sodium hydrogen carbonate, potassium citrate, potassium phosphate, potassium hydrogen phosphate, potassium hydrogen carbonate, calcium carbonate and calcium chloride and the like or combinations thereof.

In the context of the present invention, the term “co-solvent” means any solvent which is miscible in the formulation in the amount desired and which, when added provides a formulation in which the medicament can be dissolved. The function of the co-solvent is to increase the solubility of the medicament and the excipients in the formulation.

According to the present invention, the co-solvent may comprise one or more of: C2-C6 aliphatic alcohols, such as, but not limited to, ethyl alcohol and isopropyl alcohol; glycols such as but not limited to propylene glycol, polyethylene glycols, polypropylene glycols, glycol ethers; block copolymers of oxyethylene and oxypropylene; and other substances, such as, but not limited to, glycerol, polyoxyethylene alcohols, and polyoxyethylene fatty acid esters; hydrocarbons such as, but not limited, to n-propane, n-butane, isobutane, n-pentane, iso-pentane, neo-pentane, and n-hexane; and ethers such as but not limited to diethyl ether; and combinations thereof.

Suitable bulking agents may be employed in the pharmaceutical compositions of the invention, in particular aerosol compositions that are intended for administration using an MDI. The bulking agent may comprise one or more of saccharides, including monosaccharides, disaccharides, polysaccharides and sugar alcohols such as arabinose, glucose, fructose, ribose, mannose, sucrose, terhalose, lactose, maltose, starches, dextran or mannitol and the like or combinations thereof.

Suitable antioxidants that may be employed in the pharmaceutical compositions of the invention, include, but are not limited to, glycine, α-tocopherol, α-tocopherol Polyethylene Glycol Succinate (Vitamin E TPGS), ascorbic acid, propyl gallate, Butylated Hydroxy Anisole (BHA), Butylated Hydroxy Toluene (BHT), and the like or mixtures thereof;

Suitable buffers or pH adjusting agents may be employed in the pharmaceutical compositions of the invention, in particular aerosol compositions that are intended for administration using an MDI. The buffer or the pH adjusting agent may comprise one or more of organic or inorganic acids such as, but not limited to, citric acid, ascorbic acid, hydrochloric acid, sulfuric acid, nitric acid, or phosphoric acid and the like or combinations thereof.

Suitable preservatives may be employed in the pharmaceutical compositions of the invention, in particular aerosol compositions that are intended for administration using an MDI, to protect the formulation from contamination with pathogenic bacteria. The preservative may comprise one or more of benzalkonium chloride, benzoic acid, benzoates such as sodium benzoate and such other preservatives which may be known to the person having a skill in the art and combinations thereof.

Suitable complexing agents may be employed in the pharmaceutical compositions of the invention, in particular aerosol compositions that are intended for administration using an MDI, capable of forming complex bonds. The complexing agent may comprise one or more of, but not limited to, sodium EDTA or disodium EDTA and the like or combinations thereof.

In another embodiment, the pharmaceutical compositions of the present invention may be in a form suitable for intranasal delivery by nebulization.

Nebulization therapy has an advantage over other inhalation therapies, since it is easy to use and does not require co-ordination or much effort. It also works much more rapidly than medicines taken by mouth. Such compositions may comprise suitable excipients such as one or more, but not limited to, tonicity agents, pH regulators, and chelating agents in a suitable vehicle.

Suitable isotonicity adjusting agents include sodium chloride, potassium chloride, zinc chloride, calcium chloride, mannitol, glycerol, and dextrose and the like or combinations thereof.

The pH of pharmaceutical compositions of the invention may be adjusted by the addition of one or more pH regulators such as pharmacologically acceptable acids. Pharmacologically acceptable inorganic acids or organic acids may be used for this purpose. Examples of preferred inorganic acids include one or more acids selected from the group consisting of hydrochloric acid, hydrobromic acid, nitric acid, sulphuric acid, phosphoric acid and the like or combinations thereof. Examples of particularly suitable organic acids include one or more acids selected from the group consisting of ascorbic acid, citric acid, malic acid, tartaric acid, maleic acid, succinic acid, fumaric acid, acetic acid, formic acid, propionic acid and the like or combinations thereof.

Suitable chelating agents for use in pharmaceutical compositions of the invention include from salts of ethylenediaminetetraacetic acid (EDTA), such as sodium EDTA, disodium EDTA, trisodium EDTA, tetrasodium EDTA, hydroxyethylethylenediaminetriacetate (HEDTA), diethylenetriaminepentaacetate (DTPA), nitrilotriacetate (NTA), ethanoldiglycine disodium salt (EDG), diethanolglycine sodium-salt (DEG), and 1,3-propylenediaminetetraacetic acid (PDTA) or combinations thereof.

In addition to the excipients such as isotonicity-adjusting agents, pH regulators, chelating agents covered under nebulization therapy, pharmaceutical compositions for intranasal delivery in the form of a nasal spray or nasal drops may comprise thickening agents.

Suitable thickening agents for use in the pharmaceutical compositions of the invention include cellulose derivatives (for example cellulose ether) in which the cellulose-hydroxy groups are partially etherized with lower unsaturated aliphatic alcohols and/or lower unsaturated aliphatic oxyalcohols (for example methyl cellulose, carboxymethyl cellulose, hydroxypropylmethylcellulose), gelatin, polyvinylpyrrolidone, tragacanth, ethoxose (water soluble binding and thickening agents on the basis of ethyl cellulose), alginic acid, polyvinyl alcohol, polyacrylic acid, pectin and equivalent agents. Should these substances contain acid groups, the corresponding physiologically acceptable salts may also be used.

In addition to the aforementioned excipients, one or more anti-microbial preservative agents may also be added to the pharmaceutical compositions of the invention, in particular for multi-dose packages.

The pharmaceutical composition according to the present invention may be included in one or more suitable containers provided with means enabling the application of the contained formulation to the nasal mucosa or the lungs.

Where the pharmaceutical compositions of the invention are in the form of a powder for inhalation and are intended to be administered by an insufflator or powder sprayers, the pre-filled powder may be contained in a capsule, straw, tube or syringe and the like.

Insufflators, powder sprayers and powder inhalers are devices for intranasal delivery of the pharmaceutical composition of the present invention, and which may include single dose or multi-dose insufflators or powder sprayers such as, but not limited to, TriVair™ (unit-dose dry powder inhaler), OptiNose (breath-powered nasal delivery), Fit-lizer™ (multi use, single use), UniDose DP, SoluVent™, Monopowder® and the like.

Where the pharmaceutical compositions of the invention and are intended to be administered by a DPI, it may be encapsulated in capsules of gelatin or HPMC, or in blisters. The dry powder may be contained as a reservoir either in a single dose or multi-dose dry powder inhalation device. Alternatively, the powder for inhalation may be suspended in a suitable liquid vehicle and packed in an aerosol container along with suitable propellants or mixtures thereof. Alternatively, the powder for inhalation may be dispersed in a suitable gas stream to form an aerosol composition.

The pharmaceutical compositions of the invention for pulmonary delivery in the form of an aerosol composition for administration using an MDI, may be packed in plain aluminium cans or SS (stainless steel) cans or any such cans suitable for MDI delivery. Some aerosol drugs tend to adhere to the inner surfaces, i.e., walls of the cans and valves, of the MDI. This can lead to the patient getting significantly less than the prescribed amount of the active agent upon each activation of the MDI. Such cans may be suitably treated to avoid any adherence of the active on the walls thereof using techniques known in the art, for example coating the inner surface of the container with a suitable polymer can reduce this adhesion problem. Suitable coatings include fluorocarbon copolymers such as FEP-PES (fluorinated ethylene propylene and polyethersulphone) and PFA-PES (perfluoroalkoxyalkane and polyethersulphone), epoxy and ethylene. Alternatively, the inner surfaces of the cans may be anodized, plasma treated or plasma coated.

Where the pharmaceutical compositions of the invention are in the form of nasal sprays and nasal drops for administration into the nasal passages it may be done by means of a dropper (or pipette) that includes a glass, plastic or metal dispensing tube. Fine droplets and sprays can be provided by an intranasal pump dispenser or squeeze bottle as well known in the art.

The inventors of the present invention have further observed that the solubility and bioavailability properties of artemisinin derivatives were improved by nanosizing.

Nanonization of hydrophobic or poorly water-soluble drugs generally involves the production of drug nanocrystals through either chemical precipitation (bottom-up technology) or disintegration (top-down technology). Different methods may be utilized to reduce the particle size of the hydrophobic or poorly water soluble drugs. [Huabing Chen et al., discusses the various methods to develop nanoformulations in “Nanonization strategies for poorly water-soluble drugs,” Drug Discovery Today, Volume 16, Issues 7-8, April 2011, Pages 354-360].

The present invention thus provides an intrnasal pharmaceutical composition comprising an artemisinin derivative wherein the artemisinin derivative is in the nanosize range.

The term “nanosize” as used herein refers to artemisinin derivative particles having an average particle size of less than or equal to about 2000 nm, preferably less than or equal to about 1000 nm.

Mostly all particles have a particle size of less than or equal to about 2000 nm, preferably less than or equal to about 1000 nm.

The term “particles” as used herein refers to individual particle of an artemisinin derivative, or particles of artemisinin derivative granules or mixtures thereof.

The nanoparticles of the present invention may be obtained by any suitable process such as, but not limited, to milling, precipitation, homogenization, high pressure homogenization, spray-freeze drying, supercritical fluid technology, double emulsion/solvent evaporation, PRINT (Particle replication in non-wetting templates), thermal condensation, ultrasonication, spray drying. Preferably, the nanoparticles of the present invention are obtained by any nanomilling technique.

The pharmaceutical composition, according to the present invention, may further comprise at least one additional active ingredient such as, but not limited to, amodiaquine, mefloquine, lumefantrine, sulfadoxine, pyrimethamine, piperaquine, primaquine, pyronaridine, chlorproguanil, dapsone and the like or combinations thereof.

There are also provided processes for preparing the pharmaceutical composition of the present invention, which processes include, but are not limited to spray drying, dry blending and milling.

In one embodiment the pharmaceutical composition of the present invention is prepared by dissolving or dispersing the artemisinin derivative in a suitable solvent and spray drying the solution or suspension.

In another embodiment, the pharmaceutical composition of the present invention is prepared by dissolving or dispersing the artemisinin derivative in a suitable solvent, suspending the carrier in the solvent and spray drying the solution or suspension.

In yet another embodiment, the pharmaceutical composition of the present invention is prepared by dry blending the artemisinin derivative along with the carrier.

In another embodiment, the pharmaceutical composition of the present invention is prepared by milling the artemisinin derivative.

The present invention also provides a method of treating malaria by administering the pharmaceutical composition comprising an artemisinin derivative of the present invention.

The present invention also provides the pharmaceutical composition comprising an artemisinin derivative of the present invention for use in treating malaria.

The present invention also provides the use of a pharmaceutical composition comprising an artemisinin derivative of the present invention in the manufacture of a medicament for treating malaria.

The present invention also provides a method of treating malaria, wherein the method comprises administering a daily dose of from about 2 mg/kg of body weight to about 9 mg/kg of body weight to a patient.

The present invention also provides the pharmaceutical composition comprising an artemisinin derivative of the present invention for use in treating malaria by administering a daily dose of from about 2 mg/kg of body weight to about 9 mg/kg of body weight to a patient.

The present invention also provides the use of a pharmaceutical composition comprising an artemisinin derivative of the present invention in the manufacture of a medicament for treating malaria by administering a daily dose of from about 2 mg/kg of body weight to about 9 mg/kg of body weight to a patient.

The following examples are for the purpose of illustration of the invention only and are not intended in any way to limit the scope of the present invention.

### EXAMPLE 1

| Sr. No. | Ingredients | Quantity |
|---|---|---|
| 1 | Artesunate | 10% w/v |
| 2 | Ethanol | 100 ml |

Process:

1) Artesunate was dissolved in ethanol.
2) The solution obtained in step (1) was spray dried.

### EXAMPLE 2

| Sr. No. | Ingredients | Quantity |
|---|---|---|
| 1 | Artesunate | 5% w/v |
| 2 | Ethanol | 100 ml |
| 3 | Lactose | 5 |

Process:

1) Artesunate was dissolved in ethanol.
2) Lactose was suspended in the solution obtained in step (1)
3) The suspension obtained in step (2) was spray dried.

### EXAMPLE 3

| Sr. No. | Ingredients | Quantity |
|---|---|---|
| 1 | Dihydroartemisinin | 10% w/v |
| 2 | Ethanol | 100 ml |

Process:

1) Dihydroartemisinin was dissolved in ethanol.
2) The solution obtained in step (1) was spray dried.

### EXAMPLE 4

| Sr. No. | Ingredients | Quantity |
|---|---|---|
| 1 | Dihydroartemisinin | 5% w/v |
| 2 | Ethanol | 100 ml |
| 3 | Lactose | 5% w/v |

Process:

1) Dihydroartemisinin was dissolved in ethanol.
2) Lactose was suspended in the solution obtained in step (1)
3) The suspension obtained in step (2) was spray dried.

### EXAMPLE 5

| Sr. No. | Ingredients | Quantity |
|---|---|---|
| 1 | Artemisone | 10% w/v |
| 2 | Ethanol | 100 ml |

Process:

1) Artemisone was dissolved in ethanol.
2) The solution obtained in step (1) was spray dried.

### EXAMPLE 6

| Sr. No. | Ingredients | Quantity |
|---|---|---|
| 1 | Artemisone | 5% w/v |
| 2 | Ethanol | 100 ml |
| 3 | Lactose | 5% w/v |

Process:

1) Artemisone was dissolved in ethanol.
2) Lactose was suspended in the solution obtained in step (1)
3) The suspension obtained in step (2) was spray dried.

### EXAMPLE 7

| Sr. No. | Ingredients | Quantity |
|---|---|---|
| 1 | Artesunate | 2.5% w/v |
| 2 | Ethanol | 50 ml |

Process:

1) Artesunate was dissolved in ethanol.
2) Clear solution of Artesunate in ethanol obtained in step (1)
3) The solution obtained in step (2) was spray dried.

### EXAMPLE 8

| Sr. No. | Ingredients | Quantity |
|---|---|---|
| 1 | Artesunate | 2.5 gm |
| 2 | Inhalac 400 | 2.5 gm |
| 3 | Ethanol | 50 ml |

Process:

1) Artesunate was dissolved in ethanol.
2) Clear solution of Artesunate in ethanol obtained in step (1)
3) Disperse Inhalac 400 in solution of step (2)
4) The dispersion obtained in step (3) was spray dried.

### EXAMPLE 9

| Sr. No. | Ingredients | Quantity |
|---|---|---|
| 1 | Artesunate | 2.5 gm |
| 2 | Pearlitol 25C | 2.5 gm |
| 3 | Ethanol | 50 ml |

Process:

1) Artesunate was dissolved in ethanol.
2) Clear solution of Artesunate in ethanol obtained in step (1)
3) Disperse Pearlitol 25C in solution of step (2)
4) The dispersion obtained in step (3) was spray dried.

### EXAMPLE 10

| Sr. No. | Ingredients | Quantity |
|---|---|---|
| 1 | Artesunate | 2.5 gm |
| 2 | Inhalac 250 | 2.5 gm |

Process:

1) Weighed quantity of Artesunate and lactose were passed through sieve no. 60 for four passes by making bed of lactose below drug to avoid drug loss by sticking.
2) Final blend was mixed for 15 mins.

### EXAMPLE 11

| Sr. No. | Ingredients | Quantity |
|---|---|---|
| 1 | Artesunate | 2.5 gm |
| 2 | Pearlitol 50C | 2.5 gm |

Process:

1) Weighed quantity of Artesunate and Pearlitol were passed through sieve no. 60 for four passes by making bed of lactose below drug to avoid drug loss by sticking.
2) Final blend was mixed for 15 mins.

### EXAMPLE 12

| Sr. No. | Ingredients | Quantity |
|---|---|---|
| 1 | Artesunate | 2.5 gm |

Process:

1) Weighed quantity of Artesunate was milled using a jet mill to obtain an average particle size ranging from 2 μm-10 μm

### EXAMPLE 13 (Preclinical Studies)

The objective of this study was to assess the single dose pharmacokinetic profile of artesunate formulations (solution and powder) in male New Zealand White Rabbit following single intravenous injection and intranasal administration at dose of 6.25 mg/kg body weight.

### Method:

The pharmacokinetics of Artesunate formulations was assessed by intravenous and intranasal administration at dose of 6.25 mg/kg body weight using three male New Zealand Rabbits in each group. Rabbits from group 1 received intravenous injection of reference item solution through marginal ear vein. Liquid formulations of reference and test item were administered intranasally in group 2 and 3 animals respectively. Rabbits from group 4 received powder formulation of artesunate intranasally. For intranasal administration, rabbits were placed in restrainer and a light anesthesia was induced using isoflurane. After induction of anesthesia, all animals were held in a supine position during administration and for 1 min after drug administration. Blood samples were collected from group 1 at pre-dose, 0.083, 0.25, 0.5, 1.0, 2.0 and 4.0 h post dose while in group 2 to 4 blood was collected at pre-dose, 0.25, 0.5, 1.0, 2.0 and 4.0 h post dose. Plasma was obtained by centrifuging the blood samples at 5000 rpm for 10 min under 2-4° C. Separated plasma samples were analyzed for the artesunate and β-dihydroartemisinin (DHA) levels by LC-MS/MS method.

### RESULTS

No treatment related clinical signs were observed during pre- & post-dose experimental period in all the dose groups. There were no plasma concentrations observed for Artesunate in all four groups while DHA was present in quantifiable levels in plasma in all four groups.

The following is a summary of the pharmacokinetic parameters in G1, G2, G3 and G4 for DHA

| PK | G1- | G2- | G3- | G4- |
|---|---|---|---|---|
| Parameters | Artesunate | Artesunate | Artesunate | Artesunate |
| (units) | Liquid (IV) | Liquid | Liquid | Powder |
| Dose (mg/kg) | 6.25 | 6.25 | 6.25 | 6.25 |
| Tmax (h) | 0.08 ± 0.00 | 0.25 ± 0.00 | 0.25 ± 0.00 | 0.25 ± 0.00 |
| Cmax (ng/mL) | 1040.05 ± 90.23 | 119.91 ± 84.15 | 68.15 ± 17.72 | 734.28 ± 43.83 |
| | | | | *** ### |
| AUC (0-inf) | 586.62 ± 243.79 | 138.80 | 45.39 ± 4.66 | 356.97 ± 11.57 |
| (ng · h/mL) | | | | |
| AUC (0-t) | 459.42 ± 102.73 | 72.29 ± 59.51 | 40.24 ± 0.62 | 348.87 ± 10.76 |
| (ng · h/mL) | | | | *** ### |
| T1/2 (h) | 1.64 ± 1.48 | 0.33 | 0.62 ± 0.25 | 0.61 ± 0.07 |
| Bioavailability | — | 15.02 | 7.83 | 74.0 |
| (%) | | | | |

Note:
Student’s t-test was performed for group comparison of Cmax, AUC (0-t) and % F of DHA.
*** P < 0.001 as compared to Group 2 (Artesunate Liquid reference drug);
### P < 0.001 as compared to Group 3 (Artesunate Liquid formulation).

### CONCLUSION

There were no plasma concentrations observed for artesunate in all four groups while DHA was present in quantifiable levels in plasma in all four groups.

The liquid test formulation showed a decrease in the Cmax and AUC0-t of DHA as compared to the liquid reference drug formulation. In contrast, the powder test formulation showed a significant increase in the Cmax and AUC0-t. For both liquid and powder test formulations, the Tmax was found to be similar, which was equivalent to liquid reference drug formulation. Also, the elimination half life (T1/2) of both liquid and powder test formulations was found to be similar, which was higher than that of liquid reference drug formulation. These data suggest that the liquid test formulation had a higher rate of absorption, and resulted in low levels of peak plasma concentration and exposure along with slow clearance from systemic circulation and low bioavailability. The powder test formulation was quickly absorbed resulted in significantly high levels of peak plasma concentration and exposure along with slow clearance from systemic circulation and high bioavailability. This profile made the powder test formulation a better formulation than the liquid reference drug and test formulations.

Individual Plasma Concentration of Artesunate in Male NZW Rabbits at Dose of 6.25 mg/kg of Liquid reference drug Formulation by Intravenous and Intranasal Route (G1 & G2)

| | G1—Liquid reference drug Formulation— | | | *G2—Liquid reference drug Formulation— | | |
|---|---|---|---|---|---|---|
| Time | Intravenous Plasma Concentration(ng/mL) | | | Intranasal Plasma Concentration(ng/mL) | | |
| Points (h) | D031/001 | D031/002 | D031/003 | D031/004 | D031/005 | D031/006 |
| 0.0 | 0.00 | 0.00 | 0.00 | 0.00 | 0.00 | 0.00 |
| 0.083 | 0.00 | 0.00 | 0.00 | — | — | — |
| 0.25 | 0.00 | 0.00 | 0.00 | 181.154 | 0.00 | 0.00 |
| 0.5 | 0.00 | 0.00 | 0.00 | 53.747 | 0.00 | 0.00 |
| 1.0 | 0.00 | 0.00 | 0.00 | 30.173 | 0.00 | 0.00 |
| 2.0 | 0.00 | 0.00 | 0.00 | 5.686 | 0.00 | 0.00 |
| 4.0 | 0.00 | 0.00 | 0.00 | 4.717 | 0.00 | 0.00 |

* Note:
Artesunate concentration data of Rabbit 1 from G2—Intranasal Liquid formulation—reference drug (60 mg/mL) seems to be outlier.

Individual Plasma Concentration of Artesunate in Male NZW Rabbits at Dose of 6.25 mg/kg of Liquid and Powder Test Formulation by Intranasal Route (G3 & G4)

| | G3—Liquid Test Formulation— | | | G4—Powder Test Formulation— | | |
|---|---|---|---|---|---|---|
| Time | Intranasal Plasma Concentration(ng/mL) | | | Intranasal Plasma Concentration(ng/mL) | | |
| Points (h) | D031/007 | D031/008 | D031/009 | D031/010 | D031/011 | D031/012 |
| 0.0 | 0.00 | 0.00 | 0.00 | 0.00 | 0.00 | 0.00 |
| 0.083 | — | — | — | — | — | — |
| 0.25 | 0.00 | 0.00 | 0.00 | 0.00 | 0.00 | 0.00 |
| 0.5 | 0.00 | 0.00 | 0.00 | 0.00 | 0.00 | 0.00 |
| 1.0 | 0.00 | 0.00 | 0.00 | 0.00 | 0.00 | 0.00 |
| 2.0 | 0.00 | 0.00 | 0.00 | 0.00 | 0.00 | 0.00 |
| 4.0 | 0.00 | 0.00 | 0.00 | 0.00 | 0.00 | 0.00 |

Individual Plasma Concentration of DHA in Male NZW Rabbits at Dose of 6.25 mg/kg of Artesunate Liquid reference drug Formulation by Intravenous and Intranasal Route (G1 & G2)

| | G1—Liquid reference drug Formulation— | | | G2—Liquid reference drug Formulation— | | |
|---|---|---|---|---|---|---|
| Time | Intravenous Plasma Concentration(ng/mL) | | | Intranasal Plasma Concentration(ng/mL) | | |
| Points (h) | D031/001 | D031/002 | D031/003 | D031/004 | D031/005 | D031/006 |
| 0.0 | 0.00 | 0.00 | 0.00 | 0.00 | 0.00 | 0.00 |
| 0.083 | 1026.605 | 975.300 | 1136.253 | — | — | — |
| 0.25 | 354.255 | 294.694 | 571.773 | 199.006 | 129.245 | 31.484 |
| 0.5 | 218.620 | 654.96 | 185.683 | 151.059 | 63.645 | 25.429 |
| 1.0 | 252.384 | 26.433 | 51.577 | 35.403 | 26.833 | 2.584 |
| 2.0 | 137.439 | 20.211 | 0.000 | 5.986 | 0.000 | 0.000 |
| 4.0 | 0.000 | 13.606 | 0.000 | 0.000 | 0.000 | 0.000 |

Individual Plasma Concentration of DHA in Male NZW Rabbits at Dose of 6.25 mg/kg of Artesunate Liquid and Powder Test Formulation by Intranasal Route (G3 & G4)

| | G3—Liquid Test Formulation— | | | G4—Powder Test Formulation— | | |
|---|---|---|---|---|---|---|
| Time | Intranasal Plasma Concentration(ng/mL) | | | Intranasal Plasma Concentration(ng/mL) | | |
| Points (h) | D031/007 | D031/008 | D031/009 | D031/010 | D031/011 | D031/012 |
| 0.0 | 0.00 | 0.00 | 0.00 | 0.00 | 0.00 | 0.00 |
| 0.083 | — | — | — | — | — | — |
| 0.25 | 84.807 | 49.526 | 70.117 | 774.943 | 687.849 | 740.046 |
| 0.5 | 35.742 | 42.511 | 41.139 | 262.946 | 182.933 | 168.563 |
| 1.0 | 3.278 | 12.990 | 6.391 | 188.153 | 137.981 | 121.292 |
| 2.0 | 7.577 | 4.384 | 3.706 | 0.000 | 0.000 | 11.005 |
| 4.0 | 0.000 | 0.000 | 0.000 | 0.000 | 2.633 | 4.858 |

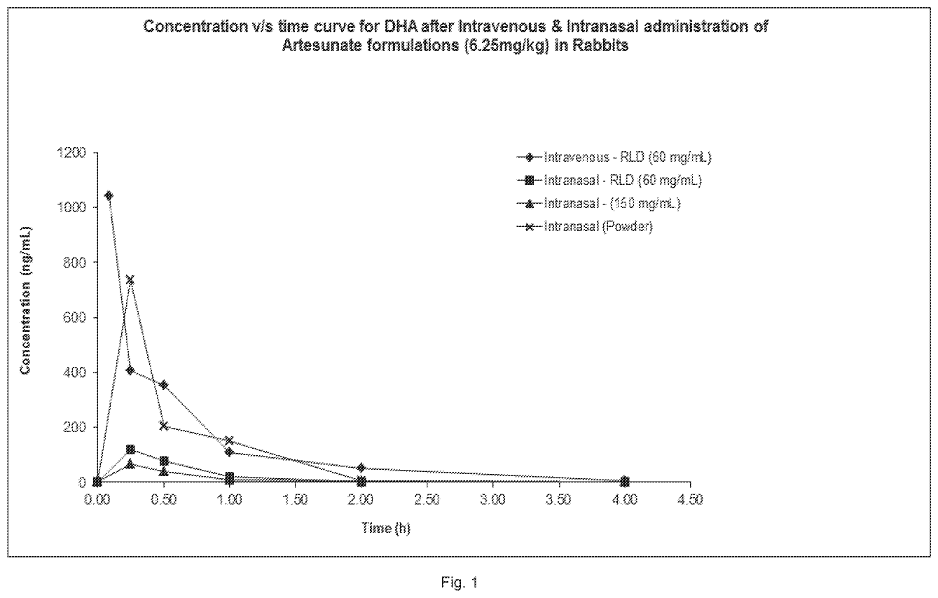

### See FIG. 1

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to be falling within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise. Thus, for example, reference to “a propellant” includes a single propellant as well as two or more different propellants; reference to a “solvent” refers to a single solvent or to combinations of two or more solvents, and the like.

1. A pharmaceutical composition for intranasal or pulmonary delivery, wherein the composition comprises an artemisinin derivative and one or more pharmaceutically acceptable excipients, wherein the one or more pharmaceutically acceptable excipients comprise a sugar alcohol, and wherein the artemisinin derivative and the sugar alcohol are provided in equal amounts.

2. A pharmaceutical composition according to claim 1, wherein the artemisinin derivative is selected from artesunate, artemether, dihydroartemisinin, artemisone, arteether, artenimol, artesunic acid, artelinic acid, deoxoartemisinin, artemotil, artemiside and their pharmaceutically acceptable salts, pharmaceutically acceptable solvates, pharmaceutically acceptable hydrates, pharmaceutically acceptable enantiomers, pharmaceutically acceptable esters, pharmaceutically acceptable polymorphs, pharmaceutically acceptable prodrugs or pharmaceutically acceptable complexes.

3. A pharmaceutical composition according to claim 1, wherein the pharmaceutical composition is for intranasal delivery and the pharmaceutical composition is in the form of a nasal spray, a solution, a suspension, nasal drops, an insufflation powder or a nasal powder.

4. A pharmaceutical composition according to claim 3, wherein the pharmaceutical composition is for intranasal delivery using a nebulizer, insufflator, powder sprayer or powder inhaler.

5. A pharmaceutical composition according to claim 3, wherein the one or more pharmaceutically acceptable excipients further comprise a carrier, a solvent, a vehicle, a thickening agent, a tonicity agent, a pH regulator, a chelating agent, or combinations thereof.

6. A pharmaceutical composition according to claim 5, wherein the solvent is selected from C2-C6 aliphatic alcohols; water, acetone, glycols, block copolymers of oxyethylene and oxypropylene; glycerol, polyoxyethylene alcohols, and polyoxyethylene fatty acid esters; hydrocarbons; ethers, or combinations thereof.

7. A pharmaceutical composition according to claim 5, wherein the thickening agent is selected from cellulose derivatives in which the cellulose-hydroxy groups are partially etherized with lower unsaturated aliphatic alcohols and/or lower unsaturated aliphatic oxyalcohols; gelatin, polyvinylpyrrolidone, tragacanth, ethoxose, alginic acid, polyvinyl alcohol, polyacrylic acid, pectin, the corresponding physiologically acceptable salts of any of the aforementioned acids, or combinations thereof.

8. A pharmaceutical composition according to claim 5, wherein the tonicity agent is selected from sodium chloride, potassium chloride, zinc chloride, calcium chloride, mannitol, glycerol, dextrose or combinations thereof.

9. A pharmaceutical composition according to claim 5, wherein the pH regulator is selected from organic or inorganic acids or combinations thereof

10. A pharmaceutical composition according to claim 5, wherein the chelating agent is selected from salts of ethylenediaminetetraacetic acid (EDTA), hydroxyethyl ethylenediaminetriacetate (HEDTA), diethylenetriaminepentaacetate (DTPA), nitrilotriacetate (NTA), ethanoldiglycine disodium salt (EDG), diethanolglycine sodium-salt (DEG), and 1,3-propylenediaminetetraacetic acid (PDTA) or combinations thereof.

11. A pharmaceutical composition according to claim 4, wherein the pharmaceutical composition is for intranasal delivery using an insufflator, a powder sprayer or a powder inhaler, and wherein the pharmaceutical composition is contained in a capsule, a straw, a tube or a syringe.

12. A pharmaceutical composition according to claim 3, wherein the pharmaceutical composition is in the form of a spray, drops, an insufflation powder or a nasal powder, and wherein the pharmaceutical composition is dispensed using a pipette, an intranasal pump dispenser or a squeeze bottle, optionally wherein the pharmaceutical composition is for pulmonary delivery using a metered dose inhaler (MDI) or a dry powder inhaler (DPI).

13. A pharmaceutical composition according to claim 1, wherein the pharmaceutical composition is for pulmonary delivery and the pharmaceutical composition is in the form of an aerosol composition or a powder.

14. A pharmaceutical composition according to claim 13, wherein the pharmaceutical composition is for pulmonary delivery using a metered dose inhaler (MDI), and wherein the one or more pharmaceutically acceptable excipients further comprise an HFC/HFA propellant, a co-solvent, a bulking agent, a non-volatile component, a buffer/pH adjusting agent, a surfactant, a preservative, a complexing agent, a vehicle, or combinations thereof.

15. A pharmaceutical composition according to claim 14, wherein the HFC/HFA propellant is selected from 1,1,1,2-tetrafluoroethane (HFA-134(a)), 1,1,1,2,3,3,3,-heptafluoropropane (HFA-227), HFC-32 (difluoromethane), HFC-143(a) (1,1,1-trifluoroethane), HFC-134 (1,1,2,2-tetrafluoroethane), HFC-152a (1,1-difluoroethane) or combinations thereof.

16. A pharmaceutical composition according to claim 14, wherein the co-solvent comprises one or more of: C2-C6 aliphatic alcohols; glycols; block copolymers of oxyethylene and oxypropylene; glycerol, polyoxyethylene alcohols, and polyoxyethylene fatty acid esters; hydrocarbons; ethers; or combinations thereof.

17. A pharmaceutical composition according to claim 14, further comprising an antioxidant selected from glycine, α-tocopherol, α-tocopherol Polyethylene Glycol Succinate (Vitamin E TPGS), ascorbic acid, propyl gallate, Butylated Hydroxy Anisole (BHA), Butylated Hydroxy Toluene (BHT), or combinations thereof.

18. A pharmaceutical composition according to claim 14, wherein the surfactant is selected from ionic and/or non-ionic surfactants, sorbitan trioleate, lecithin, isopropylmyristate, tyloxapol, polyvinylpyrrolidone, polysorbates, vitamin E-TPGS, macrogol hydroxystearates, or combinations thereof.

19. A pharmaceutical composition according to claim 14, wherein the bulking agent is selected from monosaccharides, disaccharides, polysaccharides; the sugar alcohol or another sugar alcohol selected from the group consisting of arabinose, glucose, fructose, ribose, mannose, sucrose, terhalose, lactose, maltose, starches, dextran, and combinations thereof; or combinations thereof.

20. A pharmaceutical composition according to claim 12, wherein the pharmaceutical composition is for pulmonary delivery using a dry powder inhaler (DPI) and wherein the one or more pharmaceutically acceptable excipients additionally include a carrier selected from a sugar, saccharides, amino acids, starches or starch derivatives, oligosaccharides, polyvinylpyrrolidone, alginic acid, tylose, silicic acid, organic salts, cellulose, cellulose derivatives, calcium carbonate, calcium phosphate, lactitol, dextrates, calcium stearate, dextrose, maltodextrin; magnesium stearate; cellobiose octaacetate, or combinations thereof.

21. A pharmaceutical composition according to claim 1, wherein the pharmaceutical composition comprises an anti-microbial preservative agent.

22. A pharmaceutical composition according to claim 1, wherein the artemisinin derivative is in the form of particles, and wherein the particles have a particle size of less than or equal to about 2000 nm, optionally less than or equal to about 1000 nm.

23. A pharmaceutical composition according to claim 1, wherein the composition comprises the artemisinin derivative in combination with at least one additional active ingredient, optionally wherein the at least one additional active ingredient is selected from amodiaquine, mefloquine, lumefantrine, sulfadoxine, pyrimethamine, piperaquine, primaquine, pyronaridine, chlorproguanil, dapsone or combinations thereof.

24. A pharmaceutical composition according to claim 1, wherein the sugar alcohol comprises mannitol.

25. A pharmaceutical composition according to claim 5, wherein the carrier is selected from a sugar; saccharides; amino acids; starches or starch derivatives; oligosaccharides; polyvinylpyrrolidone; alginic acid; tylose; silicic acid; organic salts; cellulose or cellulose derivatives; calcium carbonate; calcium phosphate; lactitol; dextrates; calcium stearate; dextrose; maltodextrin; magnesium stearate; cellobiose octaacetate; or combinations thereof.

26. A pharmaceutical composition according to claim 25, wherein the carrier comprises a sugar selected from the group consisting of glucose, saccharose, lactose and fructose; an amino acid selected from the group consisting of glycine, leucine, isoleucine, and arginine; an oligosaccharide selected from the group consisting of dextrins, cyclodextrins, and their derivatives; an organic salt selected from the group consisting of sodium citrate and ammonium acetate; cellulose ether; a saccharide selected from the group consisting of monosaccharides, disaccharides, and polysaccharides; the sugar alcohol or another sugar alcohol selected from the group consisting of arabinose, ribose, mannose, sucrose, trehalose, maltose, and dextran; or combinations thereof.

27. A pharmaceutical composition according to claim 6, wherein the solvent comprises a C2-C6 aliphatic alcohol selected from the group consisting of ethanol, methanol and isopropyl alcohol; a glycol selected from the group consisting of propylene glycol, polyethylene glycols, polypropylene glycols, and glycol ethers; a hydrocarbon selected from the group consisting of n-propane, n-butane, isobutane, n-pentane, iso-pentane, neo-pentane, and n-hexane; diethyl ether; or combinations thereof.

28. A pharmaceutical composition according to claim 7, wherein the thickening agent comprises a cellulose derivative selected from the group consisting of cellulose ether, methyl cellulose, carboxymethyl cellulose, and hydroxypropylmethylcellulose.

29. A pharmaceutical composition according to claim 9, wherein the pH regulator comprises an organic or inorganic acid selected from the group consisting of ascorbic acid, citric acid, malic acid, tartaric acid, maleic acid, succinic acid, fumaric acid, acetic acid, formic acid, propionic acid, hydrochloric acid, hydrobromic acid, nitric acid, sulphuric acid, phosphoric acid, and combinations thereof.

30. A pharmaceutical composition according to claim 10, wherein the chelating agent comprises a salt of ethylenediaminetetraacetic acid (EDTA) selected from the group consisting of sodium EDTA, disodium EDTA, trisodium EDTA, and tetrasodium EDTA.

31. A pharmaceutical composition according to claim 16, wherein the co-solvent comprises a C2-C6 aliphatic alcohol selected from the group consisting of ethyl alcohol and isopropyl alcohol; a glycol selected from the group consisting of propylene glycol, polyethylene glycols, polypropylene glycols, and glycol ethers; a hydrocarbon selected from the group consisting of n-propane, n-butane, isobutane, n-pentane, iso-pentane, neo-pentane, and n-hexane; diethyl ether; or combinations thereof.

32. A pharmaceutical composition according to claim 18, wherein the surfactant comprises oleic acid, polysorbate 80, macrogol-15-hydroxystearate.

33. A pharmaceutical composition according to claim 20, wherein the carrier is selected from a sugar selected from the group consisting of glucose, saccharose, lactose and fructose; an amino acid selected from the group consisting of glycine, leucine, isoleucine, and arginine; an oligosaccharide selected from the group consisting of dextrins, cyclodextrins, and their derivatives; an organic salt selected from the group consisting of sodium citrate and ammonium acetate; cellulose ether; a saccharide selected from the group consisting of monosaccharides, disaccharides, and polysaccharides; the sugar alcohol or another sugar alcohol selected from the group consisting of arabinose, ribose, mannose, sucrose, trehalose, maltose, and dextran; or combinations thereof.
